SEQUENCE LISTING (2 sequences)

SEQ ID NO: 1 - DNA, 18 bp, synthetic construct
  source 1..18: mol_type = other DNA; note = sequence of forward primer (m-Manf-F) for synthesis of Manf gene
ctgcggccaggagactgt

SEQ ID NO: 2 - DNA, 22 bp, synthetic construct
  source 1..22: mol_type = other DNA; note = sequence of reverse primer (m-Manf-F) for synthesis of Manf gene
caaccgattctctttgcctctt

(19) United States
(12) Patent Application Publication
(10) Pub. No.: US 2025/0018008 A1
(43) Pub. Date: Jan. 16, 2025

(54) USE OF MESENCEPHALIC ASTROCYTE-DERIVED NEUROTROPHIC FACTOR IN PREPARATION OF DRUG FOR TREATING HERPES SIMPLEX KERATITIS

(71) Applicant: Eye Institute of Shandong First Medical University, Qingdao (CN)

(72) Inventors: Lingling Yang, Qingdao (CN); Zongzheng Zou, Qingdao (CN); Xiaochuan Wang, Qingdao (CN); Rong Chen, Qingdao (CN); Jing Feng, Qingdao (CN); Huifeng Wang, Qingdao (CN)

(73) Assignee: Eye Institute of Shandong First Medical University, Qingdao (CN)

(21) Appl. No.: 18/523,188

(22) Filed: Nov. 29, 2023

(30) Foreign Application Priority Data
Jul. 10, 2023 (CN) 202310842375.1

(51) Int. Cl. A61K 38/17; A61P 31/22

(52) U.S. Cl. CPC A61K 38/1709; A61P 31/22

(57) ABSTRACT

Disclosed is the use of a mesencephalic astrocyte-derived neurotrophic factor (Manf) in the preparation of a drug for treating herpes simplex keratitis (HSK). Manf has an anti-herpes simplex virus type I (HSV-1) effect and can inhibit corneal vascular proliferation, corneal inflammatory response, and corneal nerve regression caused by HSV-1 infection. Manf can be used to prepare drugs for treating the HSK.

### CROSS-REFERENCE TO RELATED APPLICATION

This patent application claims the benefit and priority of Chinese Patent Application No. 202310842375.1 filed with the China National Intellectual Property Administration on Jul. 10, 2023, the disclosure of which is incorporated by reference herein in its entirety as part of the present application.

### REFERENCE TO SEQUENCE LISTING

A computer readable XML file entitled “HLP20230906866-sequence listing”, which was created on Sep. 26, 2023, with a file size of about 3,264 bytes, contains the sequence listing for this application, has been filed with this application, and is hereby incorporated by reference in its entirety.

### TECHNICAL FIELD

The present disclosure relates to the technical field of biomedicine, and in particular to the use of a mesencephalic astrocyte-derived neurotrophic factor (Manf) in the preparation of a drug for treating herpes simplex keratitis (HSK).

### BACKGROUND

Herpes simplex virus type I (HSV-1) is a double-stranded linear DNA virus with a genome length of 125 kb to 240 kb. HSV-1 is highly infectious and generally susceptible in population, mainly invading the face, brain, and parts above the waist. After infecting the human body, this virus can lie dormant in the trigeminal nerve cell bodies and remain present throughout the rest of a person's life, making it one of the most prevalent viruses in the world. HSV-1 can cause oral mucosal inflammation, cold sores, encephalitis, and other diseases. Patients infected with HSV-1 are generally accompanied by systemic symptoms, manifested in the form of clusters of small blisters. The skin and mucosal lesions of primary herpes simplex generally take 2 to 3 weeks to heal, while the skin lesions of recurrent herpes simplex mostly disappear within 1 week.

HSV-1 generally infects the skin, mucous membranes, brain, and sensory nerves, causing a variety of eye diseases such as herpes simplex of the eyelids, acute follicular conjunctivitis, dendritic keratitis, and stromal keratitis. Herpes simplex keratitis (HSK) can recur and worsen many times and may lead to blindness in severe cases. Only 1% of patients with primary HSK infection show symptoms of skin blisters, but recurrent HSK infection can cause stromal keratitis and corneal ulcers. In severe cases, corneal perforation may occur with extremely poor prognosis. Current clinical treatments for HSK mainly depend on antiviral drugs, such as acyclovir and ganciclovir. However, such antiviral drugs require long-term administration, leading to the development of drug resistance. Moreover, such drugs have a low penetration rate and are less effective in treating deep-seated viral keratitis. In addition, corticosteroids, such as glucocorticoids, are generally used clinically to treat HSK. This type of drug shows anti-inflammatory and immune response-suppressing effects, but it is not suitable for epithelial and superficial corneal inflammation, as well as inflammation with ulcers. Corticosteroid drugs have limited effects, and the abuse of hormones can aggravate corneal perforation. In summary, the treatment of HSK remains a crucial issue, and there is an urgent need to develop drugs to effectively treat HSK.

### SUMMARY

In order to solve the above problems, the present disclosure provides use of a mesencephalic astrocyte-derived neurotrophic factor in preparation of a drug for treating herpes simplex keratitis. In the present disclosure, Manf has an anti-HSV-1 effect and can inhibit reduced tear secretion and corneal sensitivity caused by corneal vascular proliferation, corneal inflammatory response, and corneal nerve regression due to HSV-1 infection. Therefore, Manf can be used to prepare drugs for treating HSK.

To achieve the above objective, the present disclosure provides the following technical solutions.

The present disclosure provides use of Manf in preparation of an HSV inhibitor.

Preferably, the HSV inhibitor has Manf at a concentration of 1 ng/ml to 100 μg/mL. Preferably, the HSV is a herpes simplex virus type I (HSV-1).

The present disclosure further provides the use of Manf in preparation of a drug for treating HSK.

Preferably, treating HSK includes one or more of inhibiting corneal vascular proliferation, inhibiting corneal inflammatory response, inhibiting corneal nerve regression, restoring corneal sensitivity, and promoting tear secretion.

Preferably, the drug for treating the HSK has Manf at a concentration of 1 ng/ml to 100 μg/mL.

### Beneficial Effects

The present disclosure provides the use of Manf in preparation of an HSV inhibitor. In the present disclosure, experiments have proved that Manf has an anti-HSV-1 effect. Manf can significantly reduce the infiltration of inflammatory factors in the cornea of HSV-1-infected mice and maintain the integrity of the corneal epithelium. Manf can directly inhibit HSV-1 infection of human corneal cells, inhibit the replication of HSV-1, and reduce the infection rate of human corneal cells. Manf can significantly restore corneal nerve regression and neovascularization in mice caused by HSV-1 infection. In summary, Manf can be used to prepare the HSV inhibitor and/or prepare the drug for treating HSK.

### BRIEF DESCRIPTION OF THE DRAWINGS

To illustrate the examples of the present disclosure or the technical solutions in the prior art more clearly, the accompanying drawings required in the examples will be briefly described below.

FIG. 1A-FIG. 1C shows expression results of Manf in normal mice and mice infected with HSV-1;

FIG. 2A-FIG. 2E show results of slit lamp photography (FIG. 2A), clinical scores of infected corneas (FIG. 2B), microscopic section results (FIG. 2C), tear secretion level (FIG. 2D), and corneal sensitivity (FIG. 2E) of mice in different treatment groups at different times;

FIG. 3A-FIG. 3B show color rendering results (FIG. 3A) and relative fluorescence intensity (FIG. 3B) of fluorescence microscopy imaging of human corneal epithelial cells in different treatment groups; where *** means there is a significant difference between a TFT group or a MANF group and a CON group; and

FIG. 4 shows photographing results by a confocal microscope in Example 4; where * indicates p<0.05, ** indicates p<0.01, and *** indicates p<0.001.

### DETAILED DESCRIPTION OF THE EMBODIMENTS

The present disclosure provides use of Manf in preparation of an HSV inhibitor.

In the present disclosure, the HSV is preferably HSV-1.

Preferably, the HSV inhibitor has Manf at a concentration of preferably 1 ng/ml to 100 μg/mL, more preferably 10 μg/mL to 20 μg/mL, and even more preferably 20 μg/mL.

In the present disclosure, Manf not only has the function of protecting cells and promoting nerve repair but also can regulate inflammation-related pathways to inhibit inflammatory responses; experiments have proved that Manf has an anti-HSV-1 effect. Manf can significantly reduce the infiltration of inflammatory factors in the cornea of infected mice and maintain the integrity of the corneal epithelium. Manf can directly inhibit HSV-1 infection of human corneal cells, and reduce the infection rate of human corneal cells. Manf can significantly restore corneal nerve regression and neovascularization in mice caused by HSV-1 infection. In summary, Manf can be used to prepare the HSV inhibitor.

The present disclosure further provides the use of Manf in preparation of a drug for treating HSK.

In the present disclosure, treating HSK includes preferably one or more of inhibiting corneal vascular proliferation, inhibiting corneal inflammatory response, inhibiting corneal nerve regression, restoring corneal sensitivity, and promoting tear secretion.

In the present disclosure, the drug for treating the HSK has Manf at a concentration of preferably 1 ng/ml to 100 μg/mL, more preferably 10 μg/mL to 20 μg/mL, and even more preferably 20 μg/mL.

In order to further describe the present disclosure, the use of Manf in preparation of the drug for treating HSK provided by the present disclosure is described in detail below with reference to the accompanying drawings and examples, but the accompanying drawings and examples should not be understood as limiting the protection scope of the present disclosure.

### Example 1

Changes of Manf in mouse corneas after HSV-1 infection

1. One (1) μL of HSV-1 (virus titer was 2×10⁸ PFU/mL) was separately injected into the corneal stroma of 12 mice. The mice were sacrificed by cervical dislocation on the first, fourth, sixth, and ninth days, respectively, and the mouse corneas were removed. Meanwhile, mice injected with 1 μL of normal saline were set as a control (CON).

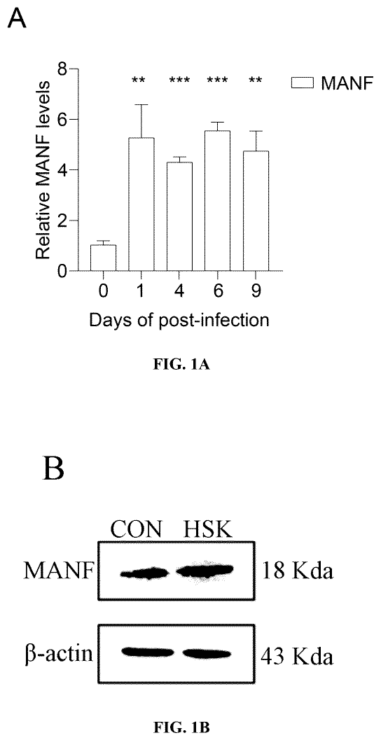

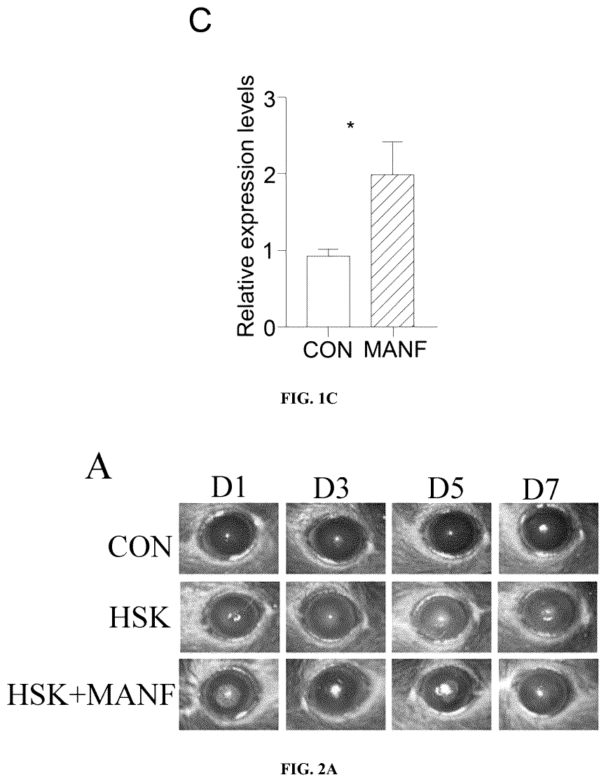

RNA from the mouse corneas was extracted using a TransZol Up Plus RNA kit (TransGen, Beijing, China). The extracted RNA was used to synthesize cDNA using a HiScript II 1st Strand cDNA synthesis kit (Epizyme Biotech, Shanghai, China). Real-time PCR was conducted with a ChamQ Universal SYBR qPCR Master Mix (Epizyme Biotech Co., Ltd., Nanjing, China) and a Rotor Gene Q Real time PCR cycler (Germany). The real-time PCR was conducted by: 95° C. for 30 sec; 95° C. for 10 sec, and 60° C. for 30 sec, a total of 40 cycles. The primers of a Manf gene were synthesized by Invitrogen Biotechnology Co., Ltd. (Shanghai, China), and specific primer sequences included: m-Manf-F: 5′-CTGCGGCCAGGAGACTGT-3′, SEQ ID NO. 1 and m-Manf-R: 5′-CAACCGATTCTCTTTGCCTCTT-3′, SEQ ID NO: 2. The mRNA levels in each sample were calculated by normalizing to mouse β-actin expression. Relative mRNA expression levels were analyzed by a 2^{-ΔΔct} method. The results are shown in FIG. 1A.

The results showed that the mRNA expression of Manf was significantly up-regulated in the cornea of mice infected with HSV-1.

2. One (1) μL of HSV-1 (virus titer was 2×10⁸ PFU/mL) was separately injected into the corneal stroma of 9 mice. The mice were sacrificed by cervical dislocation on the seventh day, and the mouse corneas were removed. A total protein (recorded as HSK) in the mouse corneal tissue was extracted with RIPA lysis buffer; at the same time, mice not injected with HSV-1 were set as a control (CON). 30 μg of protein was run on 10% SDS-PAGE gel and transferred to a polyvinylidene fluoride (PVDF) membrane (Millipore, Billerica, MA). The PVDF membrane was blocked with 5% BSA for 1 h at room temperature. After washing three times with TBST, a primary antibody Manf (dilution ratio 1:2000, Abcam, ab126321) was added; the next day, a secondary antibody (dilution ratio 1:3000; Absin Bioscience, Inc., Shanghai, China) was added to allow incubation for 2 h at room temperature. Bands were observed using a Bio-Rad Molecular Imager ChemiDoc XRS system (Bio-Rad Laboratories, Inc., Hercules, CA, USA). The β-actin was used as a control. The results are shown in FIG. 1B.

Quantitative analysis of Western blotting: After repeating the Western blotting three times, the statistical differences between an experimental group (denoted as MANF) and a control group (denoted as CON) were compared and analyzed. The results are shown in FIG. 1C.

The results showed that the expression level of Manf protein was significantly increased in the cornea of mice infected with HSV-1.

### Example 2

### Influence of Manf on HSK in Mice

1. Experimental group (denoted as HSK+MANF): 1 μL of HSV-1 (virus titer was 2×10⁸ PFU/mL) was separately injected into the corneal stroma of 13 mice; at the time when mouse cornea was infected with HSV-1, the first day after infection with HSV-1 (recorded as Day 1), the third day after infection with HSV-1 (recorded as Day 3), the fifth day after infection with HSV-1 (recorded as Day 5), and the seventh day after infection with HSV-1 (recorded as Day 7), 5 μL of 20 μg/ml Manf protein (purchased from R&D systems, Cat. No. 3748-MN) was separately injected into the subconjunctiva of mice.

Control group (recorded as HSK): similar to the experimental group, except that the mice were infected with HSV-1 but not injected with Manf protein.

On Day 1, Day 3, Day 5, and Day 7, slit lamp photos were taken of the mouse corneas, and the clinical scores of the infected corneas were evaluated. The scoring rules were as follows:

The clinical score was a score taking into account both corneal opacity and neovascularization.

The specific scores were as follows (1): 0. Normal cornea; 1. Mild corneal opacity; 2. Moderate opacity, iris visible; 3. Severe opacity, iris invisible; 4. Severe opacity with corneal ulcer;

5. Corneal perforation. Based on corneal opacity: one point was added for obvious blood vessel filling under the slit lamp; two points were added for the presence of new blood vessels from the limbus of the cornea to the pupillary limbus; and three points were added for the presence of new blood vessels in the center of the cornea.

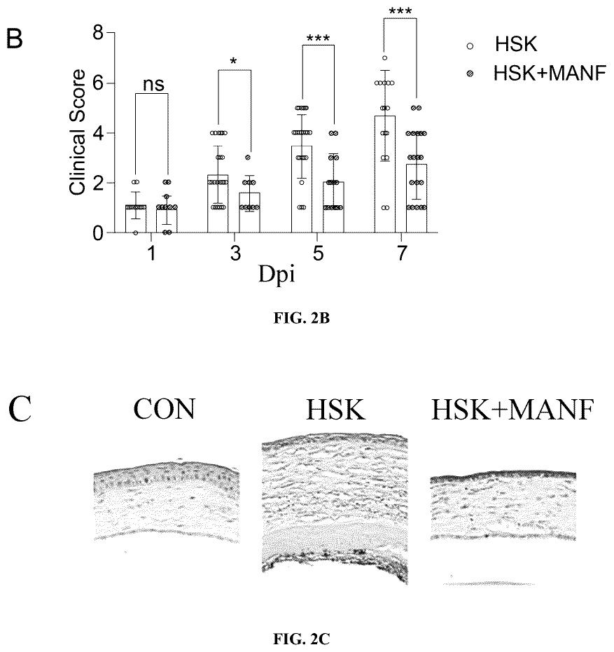

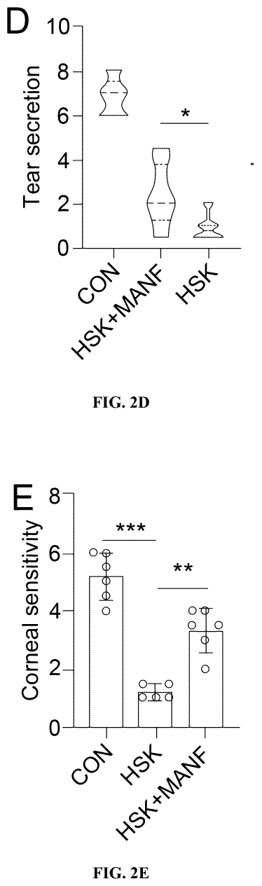

The results are shown in FIG. 2A and FIG. 2B and Table 1.

TABLE 1
Clinical scores of infected corneas of mice in different treatment groups at different times

| Group | Day 1 | Day 3 | Day 5 | Day 7 |
|---|---|---|---|---|
| HSV | 2, 1, 1, 1, 2, 1, 1, | 2, 2, 1, 3, 3, 2, 1, | 2, 3, 3, 5, 5, 3, 4, | 6, 4, 3, 4, 7, 6, 6, |
| | 1, 1, 1, 1, 1, 2, 1, | 2, 2, 1, 2, 2, 4, 2, | 4, 4, 3, 3, 4, 5, 4, | 6, 5, 6, 5, 3, 6, 4, |
| | 1, 1, 1, 1, 0, 2, 1, | 1, 3, 1, 4, 4, 1, 4, | 1, 4, 1, 4, 5, 1, 4, | 1, 6, 1, 6, 5, 1, 6, |
| | 0 | 4 | 4 | 6 |
| Average | 1.09 | 2.32 | 3.45 | 4.68 |
| score/point | | | | |
| HSV + MANF | 1, 0, 1, 1, 1, 2, 1, | 2, 2, 2, 1, 1, 2, 2, | 3, 2, 3, 2, 2, 3, 2, | 3, 4, 3, 4, 3, 4, 2, |
| | 1, 1, 1, 1, 0, 11, 0, | 2, 2, 1, 1, 1, 11, 2, | 2, 2, 1, 1, 1, 24, 1, | 5, 5, 1, 2, 3, 24, 1, |
| | 2, 1, 0, 1, 1, 2, 0, | 3, 1, 1, 1, 3, 3, 1, | 4, 1, 1, 1, 4, 4, 1, | 4, 1, 1, 1, 4, 4, 1, |
| | 1, 1, 0, 1 | 1, 2, 1, 1 | 1, 3, 1, 1 | 1, 3, 2, 2 |
| Average | 0.88 | 1.58 | 2.04 | 2.69 |
| score/point | | | | |

Note:
HSV was the control group, and a total of 22 corneas were included in the scoring; HSV + MANF was the experimental group injected with Manf protein, and a total of 26 corneas were included in the scoring; and a mean of the scores of each group was averaged.

The results showed that Manf protein could significantly reduce the symptoms of HSK in mice.

2. The experimental group and control group were set up by the same method as step 1, and mice not infected with HSV-1 were set up as blank control (marked as CON). After the injection of Manf protein on Day 7, the eyeballs of the three groups of mice were removed, dehydrated, and embedded in wax blocks. The eyeballs were sliced into 4 μm thick sections using a paraffin microtome, and the sections were baked for 30 min (until the wax melted). The slides were deparaffinized in xylene and dehydrated in alcohol. The slides were placed in a slide box containing sodium citrate buffer and heated at high pressure for 10 min to allow antigen retrieval. The slides were blocked with 10% donkey serum for 1 h. The primary antibody Manf (1:100, Abcam, ab126321) was added and incubated at 4° C. overnight. The next day, an HRP-labeled secondary antibody (1:200, Absin Bioscience) was added. Color development was performed with a DAB mixed solution. The sections were stained with hematoxylin for 3 min, rinsed with running water for 10 min to return to blue and mounted with resin. The staining results were observed under a microscope, as shown in FIG. 2C.

The results showed that Manf could significantly reduce the infiltration of inflammatory factors in the cornea of infected mice and maintain the integrity of the corneal epithelium.

3. The experimental group and control group were set up by the same method as step 1 in Example 2, and mice not infected with HSV-1 were set up as blank control (marked as CON). After the injection of Manf protein on Day 7, mice from the three groups were taken out, and the tear secretion levels and corneal sensitivity of the mice were measured.

The tear secretion level of the mice was measured using phenol red cotton thread (Tianjin Jingming Xin Technology Co., Ltd.): the lower eyelid of the mouse was exposed, and the phenol red cotton thread was quickly inserted into the mouse eyelid for 20 s; the phenol red cotton thread was removed, and a discolored length of the phenol red cotton thread was read according to the caliper. The results are shown in FIG. 2D.

Mouse corneal sensitivity was measured using a Cochet's corneal perceptometer: the mouse eyeball was completely exposed; the cornea perceptometer was regulated to an appropriate length, and then point contact was conducted on the mouse cornea; whether the mouse blinked or squinted was observed, and point contact was repeated 5 times in a row. If blinking or squinting occurred three or more times, it was considered that this length was sensitive to the mouse cornea. When the insensitive response was not measured, a length of the corneal perceptometer was recorded at this time. The results are shown in FIG. 2E.

The results showed that Manf could significantly increase the tear secretion level and corneal sensitivity of the cornea of infected mice, thereby reducing the severity of dry eye in infected mice.

### Example 3

Influence of Manf on human corneal epithelial cells infected with HSV-1

3 treatment groups: CON, TFT, and MANF were set up for the following treatments:

CON: human corneal epithelial cell lines were cultured in a medium of DMEM/F12 (Gibco) containing 10% fetal calf serum (Gibco) and penicillin-streptomycin double antibody 100 U/ml (Gibco); when a cell density was 90%, the HSV-1 with an MOI of 0.1 and a virus titer of 2×10⁶ TU/mL was added to infect human corneal epithelial cells.

Trifluorothymidine (TFT): human corneal epithelial cell lines were cultured in a medium of DMEM/F12 (Gibco) containing 10% fetal bovine serum (Gibco) and penicillin-streptomycin double antibody 100 U/ml (Gibco); when a cell density was 90%, the HSV-1 virus with an MOI of 0.1 and a virus titer of 2×10⁶ TU/mL and TFT drug (purchased from Selleck, product number S1778) with a final concentration of 50 μmol/L were added, and then co-cultured with human corneal epithelial cells.

MANF: human corneal epithelial cell lines were cultured in a medium of DMEM/F12 (Gibco) containing 10% fetal calf serum (Gibco) and penicillin-streptomycin double antibody 100 U/ml (Gibco); when a cell density was 90%, the HSV-1 with an MOI of 0.1 and a virus titer of 2×10⁶ TU/mL and the Manf protein with a final concentration of 10 μg/mL were added, and then co-cultured with human corneal epithelial cells.

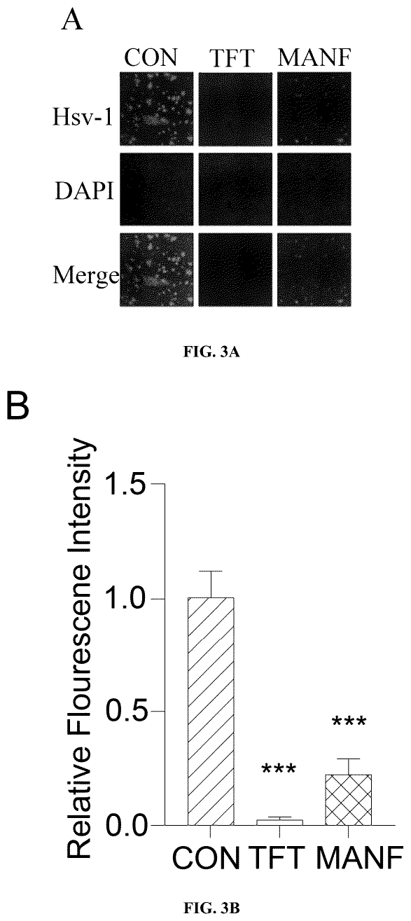

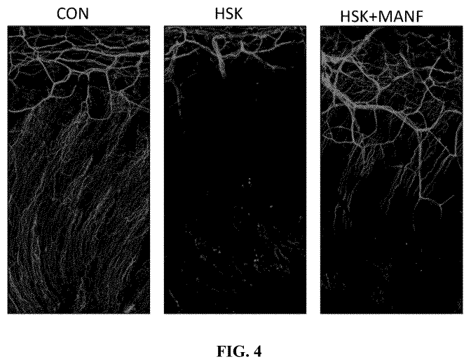

After three days of culture, the cells were fixated with 4% paraformaldehyde for 10 min, followed by permeabilization and blocking. The infected cells were stained with a GFP-HSV-1 direct-labeled antibody (Abcam, ab20437), followed by fluorescence microscopy imaging and relative fluorescence intensity measurement. The results are shown in FIG. 3.

The results showed that Manf could directly inhibit HSV-1 infection of human corneal epithelial cells.

### Example 4

3. The experimental group and control group were set up by the same method as step 1 in Example 2, and mice not infected with HSV-1 were set up as blank control (marked as CON). After injection of Manf protein on Day 7, the mice in the three groups were sacrificed, and the mouse corneas were removed. The cut corneas were placed in a 96-well plate and then permeabilized at 4° C. for 24 h in a permeabilization solution, which was a PBS solution containing 0.3% (v/v) TX-100 and 3% (v/v) BSA. A directly-labeled antibody Tubulin (Abcam, ab190575) was added to the 96-well plate and allowed to incubate at 4° C. overnight. Antibodies were prepared in a blocking solution. The primary antibody was washed with phosphate buffered saline Tween-20 (PBST) in a 48-well plate six times on a shaker for 10 min each time, where the PBST was a PBS solution containing 0.3% (v/v) TX-100 and 0.05% TW-20 (v/v). The slides were mounted in PBS containing 70% (v/v) glycerol and photographed under a confocal microscope. The results are shown in FIG. 4.

The results showed that Manf could significantly restore corneal nerve regression in mice caused by HSV-1 infection.

In conclusion, Manf has an anti-HSV-1 effect and can inhibit reduced tear secretion and corneal sensitivity caused by corneal vascular proliferation, corneal inflammatory response, and corneal nerve regression due to HSV-1 infection. Therefore, Manf can be used to prepare drugs for treating HSK.

Although the above example has described the present disclosure in detail, it is only a part of, not all of, the examples of the present disclosure. Other examples may also be obtained by persons based on the example without creative efforts, and all of these examples shall fall within the protection scope of the present disclosure.

What is claimed is:

1. A method for treating a disease caused by herpes simplex virus (HSV), comprising a step of administering an HSV inhibitor containing mesencephalic astrocyte-derived neurotrophic factor (Manf) to a subject in need thereof.

2. The herpes simplex virus inhibitor according to claim 1, wherein the HSV inhibitor contains Manf at a concentration of 1 ng/ml to 100 μg/mL.

3. The herpes simplex virus inhibitor according to claim 1, wherein the HSV is herpes simplex virus type I (HSV-1).

4. The herpes simplex virus inhibitor according to claim 2, wherein the HSV is herpes simplex virus type I (HSV-1).

5. A method for treating herpes simplex keratitis (HSK), comprising a step of administering an HSV inhibitor containing mesencephalic astrocyte-derived neurotrophic factor (Manf) to a subject in need thereof.

6. The method according to claim 5, wherein treating HSK comprises one or more of inhibiting corneal vascular proliferation, inhibiting corneal inflammatory response, inhibiting corneal nerve regression, restoring corneal sensitivity, and promoting tear secretion.

7. The method according to claim 5, wherein the drug for treating HSK contains Manf at a concentration of 1 ng/ml to 100 μg/mL.

8. The method according to claim 6, wherein the drug for treating HSK contains Manf at a concentration of 1 ng/ml to 100 μg/mL.
